(19) United States
(12) Patent Application Publication
(10) Pub. No.: US 2008/0058881 A1
(43) Pub. Date: Mar. 6, 2008

(54) METHOD AND SYSTEM FOR TREATING POST-MI PATIENTS

(75) Inventors: Darrell O. Wagner, Isanti, MN (US); Haris J. Sih, Minneapolis, MN (US); Tamara Colette Baynham, Blaine, MN (US); Steven D. Girouard, Chagrin Falls, OH (US); Joseph M. Pastore, Woodbury, MN (US); Mark Schwartz, White Bear Lake, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC, SAINT PAUL, MN (US)

(21) Appl. No.: 11/469,767

(22) Filed: Sep. 1, 2006

(51) Int. Cl. A61N 1/05

(52) U.S. Cl. 607/15; 128/903

(57) ABSTRACT

An implantable device for delivering phototherapy is described that enables the phototherapy to be delivered to internal locations in either a clinical or ambulatory setting for treatment of post-MI patients. Telemetry circuitry enables the device to deliver the phototherapy upon command or be programmed to delivery the phototherapy according to a specified schedule. The device may also incorporate one or more sensing modalities that can be used to trigger delivery of phototherapy upon occurrence of a sensed event or condition. In one particular embodiment, the phototherapy device is incorporated into a cardiac rhythm management device that also delivers pacing and/or defibrillation therapy.

### FIELD OF THE INVENTION

This invention pertains to methods and devices for treating cardiac disease.

### BACKGROUND

A myocardial infarction (MI) is the irreversible damage done to a segment of heart muscle by ischemia, where the myocardium is deprived of adequate oxygen and metabolite removal due to an interruption in blood supply. It is usually due to a sudden thrombotic occlusion of a coronary artery, commonly called a heart attack. An MI may affect the myocardium to varying degrees, ranging from relatively small infarcts to transmural or full-wall thickness infarcts, the latter occurring when a coronary artery becomes completely occluded and there is poor collateral blood flow to the affected area.

Over a period of one to two months, the necrotic tissue of the infarcted area heals, leaving fibrous scar tissue in place of the infarcted myocardium. Although the contractile function of the infarcted area is lost, surrounding myocardial fibers are usually able to compensate to an extent sufficient to permit adequate cardiac function. During period immediately after an MI and until the healing process is complete, a patient is especially vulnerable to numerous complications such as re-occlusion of a coronary artery, heart failure due to deleterious remodeling of the myocardium, and development of cardiac arrhythmias.

### BRIEF DESCRIPTION OF THE DRAWINGS

FIG. 1 illustrates the physical placement of an implantable device for delivering post-MI therapy.

FIG. 2 illustrates the functional components of an exemplary implantable device.

FIG. 3 shows a fiber optic lead for conveying light.

FIG. 4 shows a mesh-type stent having fiber optic elements embedded therein.

FIG. 5 shows a stent to which a fiber optic cable is attached.

FIG. 6 shows a lead incorporating a light-generating element.

FIG. 7 is a block diagram of a cardiac device with the capability of delivering phototherapy.

FIG. 8 is a flowchart showing the steps of an exemplary method for treatment of MI patients.

### DETAILED DESCRIPTION

Described herein are various embodiments of an implantable device and method for accelerating the healing process in post-MI patients and dealing with post-MI complications. In one embodiment, an implantable device configured to deliver phototherapy to the injured myocardium is implanted in a patient shortly after occurrence of an MI. Such phototherapy activates particular gene pathways involved in wound healing and hence accelerates healing of the myocardium after the infarct. The device may also incorporate various other functionalities to deal with common post-MI complications as well as monitoring and telemetry capabilities. The same devices and methods may also be applied to patients who are at risk of having an MI.

Previous techniques for delivering phototherapy have necessitated that it be delivered acutely in a clinical setting or only to tissues that can be reached by light transmitted to or through the skin. The implantable device for delivering phototherapy described herein enables the phototherapy to be delivered to internal locations in either a clinical or ambulatory setting. Telemetry circuitry enables the device to deliver the phototherapy upon command or be programmed to delivery the phototherapy according to a specified schedule. The device may also incorporate one or more sensing modalities that can be used to trigger delivery of phototherapy upon occurrence of a sensed event or condition. In various embodiments, the implantable device may also incorporate cardiac rhythm management functionalities such as bradycardia pacing, resynchronization pacing, myocardial stress reduction pacing for preventing cardiac remodeling, anti-tachycardia pacing, and/or defibrillation therapy. The device may also be configured to deliver drug or biological therapy in various forms which may augment the phototherapy or perform independently. The monitoring and telemetry capabilities of the device may be used to continuously monitor the patient and transmit information over a patient management network to clinical personnel in order to aid in making further treatment decisions. The device may typically be implanted in a patient at the same time coronary angioplasty and/or stent placement is performed to treat coronary stenosis. The device may then be removed after some period of time when recovery is expected to be complete (e.g., 30 days) unless there are conditions that warrant further treatment on a chronic basis.

### 1. Post-MI Phototherapy

Phototherapy or light therapy is the application of light in order to produce a photochemical or photobiological effect for the treatment of disease. Existing applications of phototherapy have involved the use of light from both coherent and non-coherent sources with wavelengths from 300 nm to 1000 nm. Red and near-infrared light is light with a wavelength of 600-1000 nm and has been found to be particularly useful in treating certain external injuries and those injuries that may be reached through the cutaneous projection of light. Red and near-infrared light is more effectively transmitted through tissue than light at shorter wavelengths, partly due to the fact that hemoglobin does not absorb strongly at these wavelengths, and has been found to be useful in treating infected, ischemic, and hypoxic wounds. The mechanisms responsible for the therapeutic benefits of phototherapy vary with the particular application. Light therapy has been found, for example, to induce the synthesis of metabolic enzymes that promote cell growth and/or proliferation, cytokines that enhance immune function, and substances that improve blood flow. The light may be delivered to either a diseased target tissue or to a target tissue that expresses factors in response to light that may be circulated away from the light source location and allowed to provide therapy elsewhere. Other types of phototherapy may involve the introduction into diseased tissue of exogenous photosensitive molecules that are then activated when light is applied.

The present disclosure relates to an implantable device for delivering light therapy to internal body locations, and in particular to infarcted myocardium. The light generated by the device may be delivered by a lead that is attached to an implantable housing and adapted to be intravascularly or otherwise internally disposed near the target tissue. An example lead has a light source such as one or more light-emitting diodes (LED's) positioned at the distal end. Another embodiment utilizes a fiber optical lead that conveys light generated by a source within the housing to the target tissue. The light source is powered by a battery (or a rechargeable power source) within the implantable housing and emits light at a specified wavelength (e.g., one between 300 nm and 1000 nm) or combination of wavelengths. The delivery of light therapy is controlled by control circuitry within the housing which, in one embodiment, is a programmable controller that can be programmed via wireless telemetry. An exemplary device thus includes an implantable lead having a light emitting structure at its distal end and connected to an implantable housing at its proximal end, a light source for generating light that is emitted by the light emitting structure of the implantable lead, control circuitry contained within the implantable housing operable to activate the light source, and a telemetry receiver interfaced to the control circuitry to enable scheduling of light activation by wireless telemetry. The phototherapy may be applied acutely where the device responds to a telemetry command to deliver therapy or chronically where the device is programmed to deliver therapy in accordance with a defined schedule or in response to sensed events. For example, the control circuitry may be programmed to activate the light source for a given length of time each day for a given number of days until such therapy is no longer required.

In one embodiment, an implantable system delivers a light therapy to promote healing of injured tissue such as that due to an MI. The implantable system emits light to induce one type of cells to produce pro-growth and/or pro-survival factors that have pro-growth and/or pro-survival effects on another type of cells. One or more light sources are positioned in locations where the pro-growth and/or pro-survival factors, after being produced, migrate to an injured region to enhance growth and regeneration of cells in that region. In one embodiment, to repair myocardial damage resulted from a myocardial infarction, a light source is positioned near tissue with fibroblast cells in a cardiovascular location upstream from the injured myocardial region. The pro-growth and/or pro-survival factors produced from the fibroblast cells are washed downstream to the injured myocardial region to enhance growth and regeneration of endogenous or transplanted stem cells in that region. To induce cells to produce pro-growth or pro-survival factors that have pro-growth or pro-survival effect in second type cells, the implantable system includes one or more light sources each emitting a light having a predetermined wavelength in a range of approximately 400 nm to 1000 nm. One example of such a light source is a red light source emitting a red light having a wavelength between 600 nm and 720 nm, with approximately 660 nm being a specific example. Another example of such a light source is an infrared light source emitting an infrared light having a wavelength between 720 nm and 1000 nm, with approximately 880 nm being a specific example. In one embodiment, the implantable system includes a plurality of light sources of the same or approximately identical wavelengths. In another embodiment, the implantable system includes a plurality of light sources emitting lights having substantially different wavelengths, such as one or more red light sources and one or more infrared light sources. The light intensity necessary to be effective depends upon the physical configuration such as the distance between the light emitting structure and the target tissue. In exemplary embodiments, the light may be delivered at intensities ranging from 1000 mcd to 10000 mcd. In various embodiments, the one or more light sources discussed above each include a light-emitting diode (LED) driven by an optical stimulation controller. The optical controller selects one or more light sources based on wavelength, controls the optical stimulation intensity by controlling an on/off state of each light source, and controls the duration of the optical stimulation by turning each light source on and off. In various embodiments, the present subject matter is generally applicable to healing of injured cardiac and non-cardiac tissues.

### 2. Implantable Device Description

Internal phototherapy may be delivered by an implantable device dedicated to that purpose or configured to also deliver other cardiac therapies such as bradycardia pacing, cardioversion/defibrillation therapy, cardiac resynchronization therapy, or drug delivery. The physical configuration and implantation technique for the device are similar to that of conventional cardiac pacemakers and implantable cardioversion/defibrillation devices. Implantable devices such as pacemakers and cardioverter/defibrillators are battery-powered devices which are usually implanted subcutaneously on the patient's chest and connected to electrodes by leads threaded through the vessels of the upper venous system into the heart.

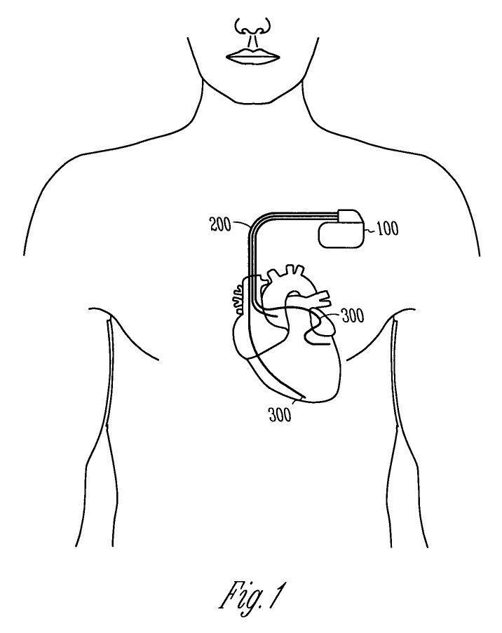

FIG. 1 shows an implantable device 100 for delivering phototherapy that is adapted to be placed subcutaneously or submuscularly in a patient's chest with one or more leads 200 extending therefrom that are threaded intravenously into the heart. At the distal end of the leads 200 are light-emitting structures 300 used to deliver phototherapy to cardiac tissue. In the figure, one of the light-emitting structures 300 is disposed in the right ventricle while the other is disposed in a cardiac vein so as to be in contact with the left ventricle. The light-emitting structure 300 may be one or more light-emitting diodes actuated and powered by a conductor within the lead 200 or may be the end of a fiber optic cable within the lead that is used to transmit light generated by one or more light-emitting diodes in the implantable device 100. In either case, the one or more light-emitting diodes may be designed to emit light at wavelengths ranging from 400 to 1000 nm. For an example, a lead could have both blue (470 nm) and red (630 nm) LED light sources. Such a lead would allow the blue light to illuminate the heart without being absorbed as much as would be the case using a vasculature approach. The device could control the wavelengths separately as predetermined for best therapy.

The leads 200 may also include conventional leads that connect the device to electrodes used for sensing cardiac activity and for delivering electrical stimulation (i.e., either pacing pulses or defibrillation shocks) to the heart. As aforesaid, the light emitted by the implantable phototherapy device is used to improve the healing process of cells in the region of a myocardial infarction (MI). For an MI located at the cardiac apex, for example, a phototherapy lead may be placed in the great cardiac vein and positioned near the apex so that light radiates into the region of the MI. Prophylactic cardioprotective therapy can be delivered periodically (e.g., every 24-72 hr) or acutely during reperfusion in scheduled revascularization therapies. In addition to delivering scheduled and on-demand phototherapy, an implantable cardiac device may also incorporate functionality for delivering phototherapy upon occurrence of particular events or when particular conditions are determined to be present.

In another embodiment, a lead having a light source is designed to approach the heart epicardially percutaneously through the chest wall. The light source can then be positioned where it illuminates the region of infarct injury. This can be especially useful for light sources having wavelengths less than 600 nm that do not transmit well through blood and tissue. The epicardial lead may contain pacing and defibrillation capibilities or be a separate lead from those in the vasculature having such capibilities. Instead of a lead, the light source could also transmit light through a fiberoptic cable designed to approach the heart epicardially percutaneously through the chest wall.

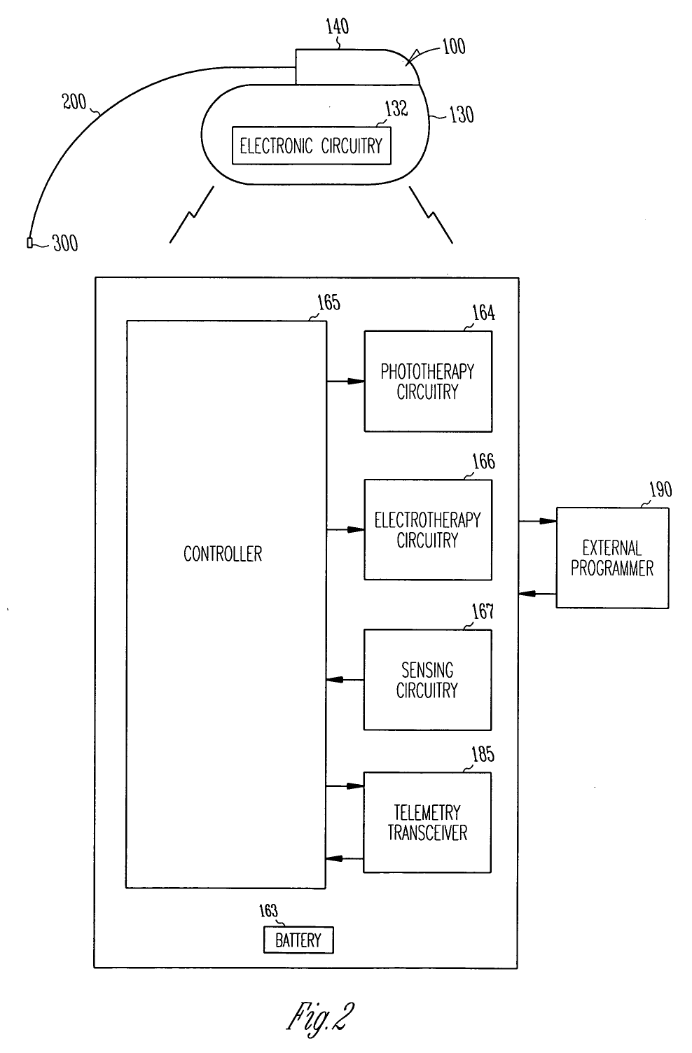

FIG. 2 illustrates the implantable device 100 for delivering internal phototherapy in more detail. The device 100 includes a hermetically sealed housing 130, formed from a conductive metal, such as titanium, which may also serve as an electrode for sensing or electrical stimulation. A header 140, which may be formed of an insulating material, is mounted on housing 130 for receiving a lead 200 used to deliver phototherapy to tissues in proximity to the light-emitting structure 300 at the end of the lead. The header also receives leads for cardiac sensing and stimulation if the device also incorporates that functionality. Contained within the housing 130 is the electronic circuitry 132 for providing the light generating functionality to the device as described herein and, in the case of a pacemaker or cardioverter/defibrillator, the circuitry for sensing and electrically stimulating the heart. The electronic circuitry 132 includes a controller 165 that may be made up of discrete circuit elements but is preferably a processing element such as a microprocessor together with associated memory for program and data storage which may be programmed to perform algorithms for delivering therapy and monitoring physiological parameters. The controller 165 controls the operation of phototherapy circuitry 164 which either comprises one or more light-generating elements (e.g., a light-emitting diode) or circuitry for actuating one or more light-generating elements at the end of the lead 200. A battery 163 provides power for the light-generating element as well as the rest of the electronic circuitry 132. A telemetry receiver or transceiver 185 capable of wirelessly communicating with an external device 190 is also interfaced to the controller 165. The external device 190 may be an external programmer that wirelessly communicates with the device 100 and enables a clinician to issue commands to the implantable device and modify the programming of the controller. The device thus delivers phototherapy under programmed control as implemented in the programming of the controller 165 and may deliver such therapy at programmed times and for programmed durations, in response to sensed conditions or events, or upon receiving a command to do so via telemetry. The external device 190 shown in the figure may also be a remote monitoring unit that may be interfaced to a patient management network enabling the implantable device to transmit data and alarm messages to clinical personnel over the network as well as be programmed remotely. The network connection between the external device 190 and the patient management network may be implemented by, for example, an internet connection, over a phone line, or via a cellular wireless link.

In addition to delivering phototherapy, the device 100 may also be configured as a pacemaker capable of delivering bradycardia and/or antitachycardia pacing, an implantable cardioverter/defibrillator, a combination pacemaker/defibrillator, a drug delivery device, or a monitoring-only device. The device 100 may be equipped for these purposes with one or more leads with electrodes for disposition in the right atrium, right ventricle, in a cardiac vein for sensing cardiac activity and/or delivering electrical stimulation to the heart, or be adapted for intra-vascular or other disposition in order to provide other types of sensing functionality. Also shown as interfaced to the controller 165 in FIG. 2 are electrotherapy circuitry 166 for delivering electrical stimulation and sensing circuitry 167 for detecting cardiac activity as well as measuring values of other physiological parameters. For example, the sensing circuitry may include an accelerometer, a minute ventilation sensor, a trans-thoracic impedance sensor, an acoustic sensor, and/or a temperature sensor.

In different embodiments, a lead for delivering phototherapy may convey light generated by circuitry within the implantable device housing or may be used to control the operation of a light-generating element attached to the lead. FIG. 3 illustrates an example of the first type of embodiment in which a lead 350 comprises a sheath 351 that surrounds a fiber optic cable 352. At the proximal end of the lead 350 is an optical coupler 354 that attaches to a light conveying structure (e.g., a fiber optic cable) in the header 140 of the device in order to receive light produced by the light generating element of phototherapy circuitry 164. The light is then delivered out of the distal end 358 of the lead 350 to a selected site. The distal end of the lead may be left free to float in a blood vessel or heart chamber or may be connected to a stent or similar structure. In one specific example, the lead 350 includes a plurality of fiber optic cables each having an optical coupler at the proximal end to attach to a light conveying structure in the header 140 of the device in order to receive light produced by the light generating element of phototherapy circuitry 164. The fiber optic cables each have a distal end being a light-emitting site on the lead 350. The coupler couplers may attach to light generating elements with substantially different wavelengths such as LED's that emit at different wavelengths. The light-emitting sites may be arranged to emit lights in substantially the same or different directions. The phototherapy circuitry 164 may turn each light-generating source on and off to control the wavelength, intensity, and timing of the light therapy.

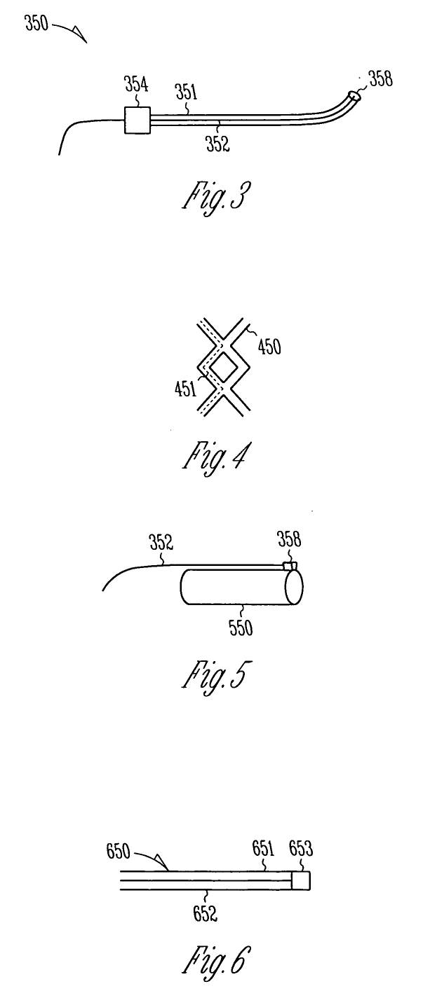

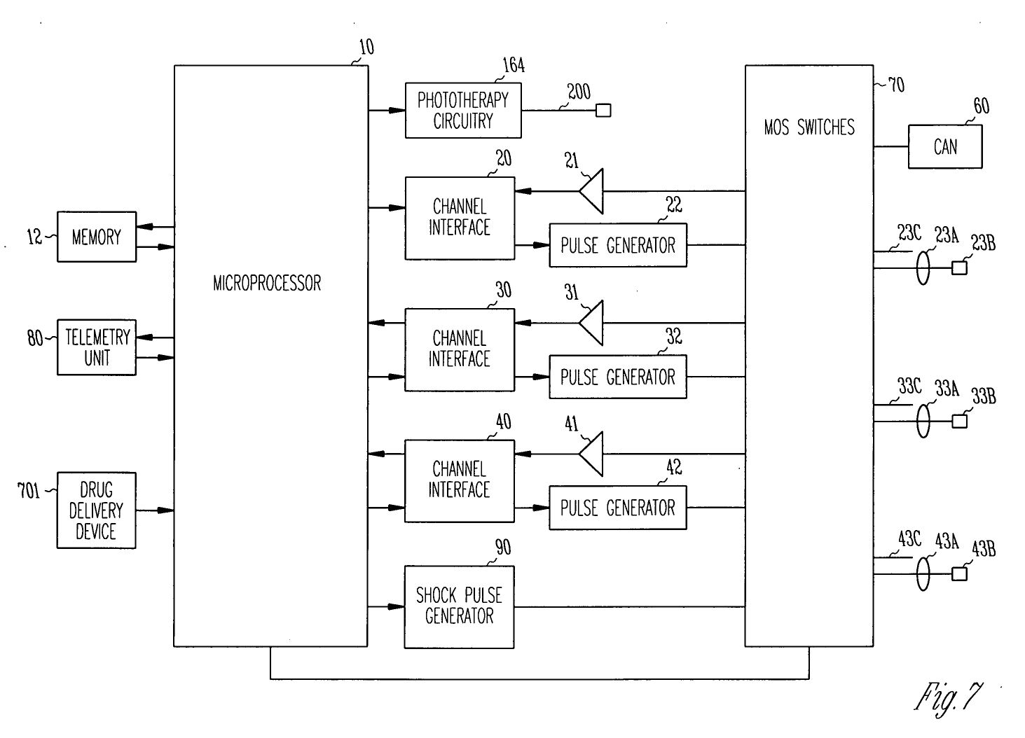

FIG. 4 illustrates an embodiment of a mesh-type stent 450 that has optical elements 451 embedded (e.g., by photolithographic methods) within its structure to which the fiber optic cable 352 is optically coupled. FIG. 5 illustrates another embodiment in which the distal portion of the fiber optic cable 352 is incorporated in a stent body 550. FIG. 6 illustrates another embodiment of a lead for delivering phototherapy in which a lead 650 comprises one or more electrical conductors 652 within a sheath 651 and a light-generating element 653 (e.g., a light emitting diode) at the distal end of the lead. The light-generating element 653 is powered and controlled via the conductors 652 which are connected to the phototherapy circuitry 164 of the implantable device. In a specific example, lead 650 includes a plurality of light-generating elements (e.g., light emitting diodes) at and near the distal end of the lead. The light-generating elements are powered and controlled via conductors connected to the phototherapy circuitry 164 of the implantable device. The light-generating elements may have approximately identical wavelengths or substantially different wavelengths, and may be arranged along lead 650 to emit light in substantially the same or different directions. The phototherapy circuitry 164 may turn each light-generating source on and off to control the wavelength, intensity, and timing of the light therapy.

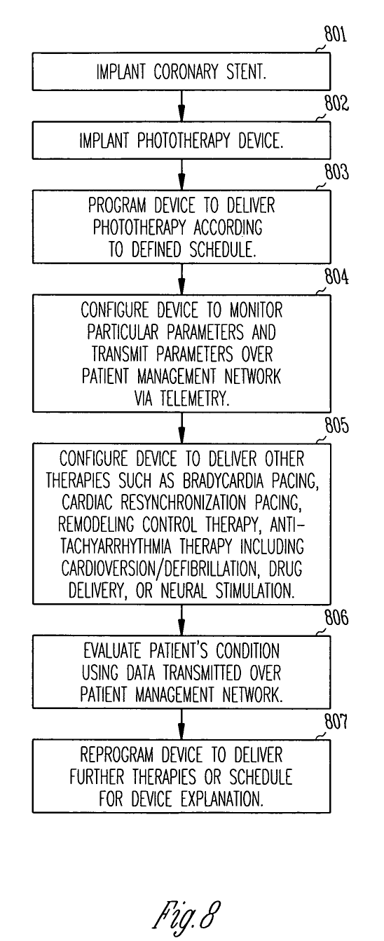

FIG. 7 is a block diagram of an implantable phototherapy device with cardiac sensing, pacing, and defibrillation capability and which may be programmed to delivery phototherapy when certain events or conditions are detected. The controller of the device is made up of a microprocessor 10 communicating with a memory 12 via a bidirectional data bus, where the memory 12 typically comprises a ROM (read-only memory) for program storage and a RAM (random-access memory) for data storage. The controller is capable of operating the device so as to deliver a number of different therapies in response to detected cardiac activity. The controller is interfaced to phototherapy circuitry 164, which may include an LED light source, for controlling the delivery of phototherapy through phototherapy lead 200. A telemetry unit 80 is also provided for enabling the controller to communicate with an external programmer or other device via a wireless telemetry link.

The device shown in FIG. 7 has three sensing/pacing channels, where a pacing channel is made up of a pulse generator connected to an electrode while a sensing channel is made up of the sense amplifier connected to an electrode. A MOS switch matrix 70 controlled by the microprocessor is used to switch the electrodes from the input of a sense amplifier to the output of a pulse generator. The switch matrix 70 also allows the sensing and pacing channels to be configured by the controller with different combinations of the available electrodes. A shock pulse generator 90 is also interfaced to the controller for delivering defibrillation shocks between an electrode and the housing or can 60 as selected by the switch matrix. In an example configuration, a sensing/pacing channel may include ring electrode 43a (33a or 23a) and tip electrode 43b (33b or 23b) of bipolar lead 43c (33c or 23c), sense amplifier 41 (31 or 21), pulse generator 42 (32 or 22), and a channel interface 40 (30 or 20). The channel interfaces communicate bi-directionally with a port of microprocessor 10 and may include analog-to-digital converters for digitizing sensing signal inputs from the sensing amplifiers, registers that can be written to for adjusting the gain and threshold values of the sensing amplifiers, and registers for controlling the output of pacing pulses and/or changing the pacing pulse amplitude. In the illustrated embodiment, the device is equipped with bipolar leads that include two electrodes which are used for outputting a pacing pulse and/or sensing intrinsic activity. Other embodiments may employ unipolar leads with single electrodes for sensing and pacing which are referenced to the device housing or can 60 (or another electrode) by the switch matrix 70. The channels may be configured as either atrial or ventricular channels so as to enable either bi-atrial or biventricular pacing. For example, a configuration for biventricular sensing/pacing could have one lead of a channel disposed in the right ventricle for right ventricular sensing/pacing and another lead of a channel disposed in the coronary sinus for left ventricular sensing/pacing. By appropriate lead placement and adjustment of pulse parameters, a pacing channel may also be configured to deliver neural stimulation such as stimulation of the vagus nerve.

The controller controls the overall operation of the device in accordance with programmed instructions stored in memory, including controlling the delivery of paces via the pacing channels, interpreting signals received from the sensing channels, implementing timers, and delivering defibrillation shocks. The sensing circuitry of the pacemaker detects a chamber sense when an electrogram signal (i.e., a voltage sensed by an electrode representing cardiac electrical activity) generated by a particular channel exceeds a specified intrinsic detection threshold. A chamber sense may be either an atrial sense or a ventricular sense depending on whether it occurs in the atrial or ventricular sensing channel. By measuring the intervals between chamber senses, the device is able to determine an atrial or ventricular rate, and pacing algorithms used in particular pacing modes employ such senses to trigger or inhibit pacing. Measured atrial and ventricular rates are also used to detect arrhythmias such as fibrillation so that a defibrillation shock can be delivered if appropriate.

Also shown in FIG. 7 as interfaced to the controller is a drug delivery device 701 that may be employed in conjunction with phototherapy or used to treat other conditions independently. In one embodiment, the drug delivery device includes a drug reservoir and pumping apparatus within the implantable device that delivers the drug through a lumen in one of the implanted leads. In another embodiment, a stent similar to that described above for delivering phototherapy may incorporate a drug container with elution of the drug controlled by a signal transmitted from the implantable device via an attached lead. The drug delivered by the drug delivery device may be photoactive substances that are activated by the light delivered from the device or other substances that act in conjunction with the phototherapy to promote healing. The device may also be configured to deliver drugs to treat other conditions likely to arise in a post-MI patient such as cardiac ischemia and cardiac arrhythmias.

In one embodiment, the device of FIG. 7 is programmed to deliver phototherapy to the heart subsequent to delivery of a defibrillation shock. When ventricular fibrillation occurs, the myocardium can become stunned which can lead to myocardial dysfunction for some time even after normal rhythm is restored. Myocardial stunning is this situation may be due at least partly to the coronary blood flow being compromised during ventricular fibrillation. The device may be programmed to deliver phototherapy after an episode of ventricular fibrillation in effort to mitigate this phenomenon. Because the untoward aftereffects of ventricular fibrillation may vary with the duration of the ventricular fibrillation, the device may be further programmed to only deliver phototherapy when the episode of ventricular fibrillation has lasted for a specified duration and/or required a specified number of defibrillation shocks before being terminated.

As noted above, light therapy can be beneficial in allowing myocardial regions that have been injured due to ischemia to heal. Light therapy may also be beneficial in preventing or reducing the reperfusion injury that occurs when blood flow is restored to the myocardium after an ischemic event. The device may also be configured to detect cardiac ischemia using its sensing channels and deliver phototherapy accordingly. In order to detect whether the patient is experiencing cardiac ischemia, the controller is programmed to analyze the recorded electrogram of an evoked response or intrinsic beat and look for a “current of injury.” When the blood supply to a region of the myocardium is compromised, the supply of oxygen and other nutrients can become inadequate for enabling the metabolic processes of the cardiac muscle cells to maintain their normal polarized state. An ischemic region of the heart therefore becomes abnormally depolarized during at least part of the cardiac cycle and causes a current to flow between the ischemic region and the normally polarized regions of the heart, referred to as a current of injury. A current of injury may be produced by an infarcted region that becomes permanently depolarized or by an ischemic region that remains abnormally depolarized during all or part of the cardiac cycle. A current of injury results in an abnormal change in the electrical potentials measured by either a surface electrocardiogram or an intracardiac electrogram. If the abnormal depolarization in the ventricles lasts for the entire cardiac cycle, a zero potential is measured only when the rest of the ventricular myocardium has depolarized, which corresponds to the time between the end of the QRS complex and the T wave in an electrogram and is referred to as the ST segment. After repolarization of the ventricles, marked by the T wave in an electrogram, the measured potential is influenced by the current of injury and becomes shifted, either positively or negatively depending upon the location of the ischemic or infarcted region, relative to the ST segment. Traditionally, however, it is the ST segment that is regarded as shifted when an abnormal current of injury is detected by an electrogram or electrocardiogram. A current injury produced by an ischemic region that does not last for the entire cardiac cycle may only shift part of the ST segment, resulting in an abnormal slope of the segment. A current of injury may also be produced when ischemia causes a prolonged depolarization in a ventricular region which results in an abnormal T wave as the direction of the wave of repolarization is altered.

In order to detect a change in an electrogram indicative of ischemia, a recorded electrogram is analyzed and compared with a reference electrogram, which may either be a complete recorded electrogram or particular reference values representative of an electrogram. Because certain patients may always exhibit a current of injury in an electrogram (e.g., due to CAD or as a result of electrode implantation), the controller is programmed to detect ischemia by looking for an increased current of injury in the recorded electrogram as compared with the reference electrogram, where the latter may or may not exhibit a current of injury. One way to look for an increased current of injury in the recorded electrogram is to compare the ST segment amplitude and/or slope with the amplitude and slope of a reference electrogram. Various digital signal processing techniques may be employed for the analysis, such as using first and second derivatives to identify the start and end of an ST segment. Other ways of looking for a current injury may involve, for example, cross-correlating the recorded and reference electrograms to ascertain their degree of similarity. The electrogram could be implicitly recorded in that case by passing the electrogram signal through a matched filter that cross-correlates the signal with a reference electrogram. The ST segment could also be integrated, with the result of the integration compared with a reference value to determine if an increased current of injury is present. If a change in a recorded electrogram indicative of ischemia is detected, the device delivers phototherapy to the myocardium for a specified duration. The device may be further programmed to only deliver the light therapy if the ischemic indication has persisted for a specified length of time.

### 3. Exemplary Method for Treating MI

FIG. 8 shows the steps involved in an exemplary method for treating a post-MI patient employing an implantable device as described herein. After initial evaluation and stabilization of the patient after an MI, a coronary stent may be implanted as shown at step 801. This may present a convenient opportunity to implant the phototherapy device at step 802. At step 803, the device is programmed to deliver phototherapy to one or more selected myocardial regions according to a defined schedule. The device could also be programmed to deliver such phototherapy in response to particular sensed conditions or to commands received via telemetry. At step 804, the device is configured to monitor particular physiological parameters and transmit those parameters over a patient management network via telemetry to for evaluation by clinicians. At step 805, the device may be programmed to deliver other therapies such as bradycardia pacing, cardiac resynchronization pacing, remodeling control therapy, anti-tachyarrhythmia therapy including cardioversion/defibrillation, drug delivery, or neural stimulation. For example, certain post-MI patients may suffer from conduction deficits either temporarily or chronically so that they would benefit from cardiac resynchronization pacing delivered, for example, as left ventricle-only pacing or biventricular pacing. The device could also be configured to deliver pacing to one or more sites using a bradycardia pacing mode that pre-excites one or more areas of the myocardium relative to other areas during systole. Such pre-excitation of those areas subjects them to lessened mechanical stress in order to help prevent the deleterious cardiac remodeling that commonly occurs in post-MI patients. At step 806, the patient's condition is evaluated using the data transmitted over patient management network. At step 807, the device may then be programmed to deliver further therapies, or the patient may be scheduled for device explantation.

Although the invention has been described in conjunction with the foregoing specific embodiment, many alternatives, variations, and modifications will be apparent to those of ordinary skill in the art. Such alternatives, variations, and modifications are intended to fall within the scope of the following appended claims.

What is claimed is:

1. A method for treating a patient, comprising:
implanting an implantable phototherapy device;
configuring and programming the device to deliver phototherapy to one or more infarcted areas of the patient's myocardium;
configuring the device to monitor one or more physiological parameters and to transmit those parameters over a patient management network via telemetry;
evaluating the patient using the transmitted physiological parameters; and,
reprogramming the device to deliver further therapies or scheduling the patient for device explantation.

2. The method of claim 1 further comprising configuring and programming the device to deliver cardioversion/defibrillation shock therapy in response to detected tachyarrhythmias.

3. The method of claim 1 further comprising configuring and programming the device to deliver a photoactive drug in conjunction with the phototherapy.

4. The method of claim 1 further comprising configuring and programming the device to deliver pre-excitation pacing to one or more myocardial sites in order to help prevent cardiac remodeling.

5. The method of claim 1 further comprising configuring and programming the device to deliver cardiac resynchronization pacing.

6. The method of claim 1 further comprising configuring the device to deliver phototherapy via a fiber optic lead placed in the patient's heart.

7. The method of claim 1 further comprising configuring the device to deliver phototherapy via a light emitting source at the distal end of a lead placed in the patient's heart.

8. The method of claim 1 further comprising implanting a stent for delivering phototherapy.

9. The method of claim 1 further comprising configuring and programming the device to detect cardiac ischemia and to deliver one or more therapies in response thereto.

10. The method of claim 1 further comprising configuring and programming the device to deliver neural stimulation.

11. A system, comprising:
an implantable device that includes an implantable housing and a lead having a light emitting structure at its distal end and connected to the implantable housing at its proximal end;
a light source for generating light that is emitted by the light emitting structure of the implantable lead;
control circuitry contained within the implantable housing operable to activate the light source and deliver phototherapy;
sensing circuitry contained within the implantable housing and one or more leads attached thereto for monitoring one or more physiological parameters;
a telemetry transceiver interfaced to the control circuitry to enable communication with the device by wireless telemetry, wherein the device may be programmed to deliver phototherapy according to a defined schedule; and,
a remote monitoring unit for receiving the one or more physiological parameters monitored and transmitted by the device and transmitting the received parameters over a patient management network.

12. The system of claim 11 wherein the light source of the implantable device is contained within the implantable housing and conveys light to the light emitting structure through an optical fiber within the implantable lead.

13. The system of claim 11 wherein the light source of the implantable device is contained within the distal portion of the implantable lead.

14. The system of claim 11 wherein the light source of the implantable device is a light emitting diode.

15. The system of claim 11 wherein the control circuitry of the implantable device is programmed to deliver light therapy at periodic intervals.

16. The system of claim 11 wherein the implantable device further comprises:
one or more leads with electrodes for generating electrogram signals produced by cardiac activity;
sensing circuitry contained within the implantable housing for receiving the electrogram signals; and,
wherein the control circuitry is programmed analyze the electrogram signals and to deliver light therapy if a current of injury indicative of cardiac ischemia is detected.

17. The system of claim 16 wherein the control circuitry of the implantable device is further programmed to deliver light therapy only if the ischemic indication has persisted for a specified length of time.

18. The system of claim 11 wherein the implantable device further comprises:
one or more leads with electrodes for generating electrogram signals produced by cardiac activity;
sensing circuitry contained within the implantable housing for receiving the electrogram signals;
a shock generator and a lead for delivering a defibrillation shock;
wherein the control circuitry is programmed to cause delivery of a defibrillation shock upon detection of ventricular fibrillation from the electrogram signals and to cause delivery of light therapy subsequent to termination of the ventricular fibrillation.

19. The system of claim 18 wherein the control circuitry of the implantable device is further programmed to deliver light therapy subsequent to termination of the ventricular fibrillation only if the fibrillation has lasted for a specified length of time.

20. The system of claim 18 wherein the control circuitry of the implantable device is further programmed to deliver light therapy subsequent to termination of the ventricular fibrillation only if a specified number of defibrillation shocks were delivered.
